(19) United States
(12) Patent Application Publication
(10) Pub. No.: US 2016/0220692 A1
(43) Pub. Date: Aug. 4, 2016

(54) TARGETING THE M2-TUMOR ASSOCIATED MACROPHAGE FOR CANCER THERAPY

(71) Applicant: THE JOHNS HOPKINS UNIVERSITY, Baltimore, MD (US)

(72) Inventor: Kenneth PIENTA, Baltimore, MD (US)

(21) Appl. No.: 14/917,744

(22) PCT Filed: Sep. 9, 2014
(86) PCT No.: PCT/US2014/054731

§ 371 Date: Mar. 9, 2016

Related U.S. Application Data
(60) Provisional application No. 61/875,300, filed on Sep. 9, 2013.

(51) Int. Cl. A61K 47/48; C07K 16/30; A61K 38/08; C07K 16/28

(52) U.S. Cl. CPC A61K 47/48561; C07K 16/2851; C07K 16/30; A61K 47/48569; A61K 38/08; A61K 47/48884

(57) ABSTRACT

The present invention features methods of directly targeting specific cell surface receptors on the M2 macrophage for antibody or nanoparticle directed therapy.

### CROSS REFERENCE TO RELATED APPLICATIONS

This application is related to U.S. Provisional Patent Application Ser. No. 61/875,300, filed Sep. 9, 2013. The entire contents of this patent application are hereby incorporated by reference herein.

### BACKGROUND OF THE INVENTION

Malignant tumors are associated with an immune infiltrate as part of the reactive stroma that is enriched for macrophages (1-7). Macrophages also play an important role in the regulation of angiogenesis in both normal and diseased tissues, including malignant tumors (7-9). While it is not clear whether tumor associated macrophages (TAMs) are derived from peripheral blood monocytes recruited into the tumor from the circulation or from resident macrophages already in the healthy tissue before tumor develops/metastasizes, their importance in facilitating tumor growth by promoting neovascularization and matrix degradation is well documented (10). Elevated expression of a number of monocyte chemoattractants, including CCL2, CCL3, CCL4, CCL8 and CCL5 (RANTES) by both tumor and stromal cells within tumors has been shown to positively correlate with increased

TAM numbers in many human tumors (11-14). When associated with tumors, macrophages demonstrate functional “polarization” towards one of two phenotypically different subsets of macrophages: TH1 (also known as M1 macrophages) or TH2 (also known as M2 macrophages) (14). M1 macrophages are known to produce pro-inflammatory cytokines and play an active role in cell destruction while M2 macrophages primarily scavenge debris and promote angiogenesis and wound repair (2-14). TAMs are known to be important for tumor growth. TAMs originate from circulating monocytes and their recruitment into tumors is driven by tumor-derived chemotactic factors. TAMs promote tumor cell proliferation and metastasis by secreting a wide range of growth and proangiogenic factors. Consequently, many tumors with a high number of TAMs have an increased tumor growth rate, local proliferation and distant metastasis. The M2 macrophage population is phenotypically similar to the TAM population that promotes tumor growth and development.

TAMs have been demonstrated to make up to 50% of the population of cells in PCa bone metastases, contributing to cancer cell growth by promoting a permissive growth environment through the secretion of matrix degrading enzymes, angiogenic factors, and multiple growth factors (1-18). In addition, recent evidence has demonstrated that M2-TAMs induce epithelial cancer cells to undergo an epithelial to mesenchymal transition (EMT) promoting metastasis (19). It has previously been demonstrated that inhibiting the accumulation of M2-TAMs effectively blocked prostate cancer tumor growth (20) Inhibition of this accumulation by blocking the chemoattractant CCL2 was ineffective in clinical trials because the antibody used was not effective in blocking free CCL2 and macrophages still accumulated in the tumors (21).

Therefore, directly targeting M2-TAMs to treat neoplasms represents an underdeveloped frontier in cancer therapeutics (22,23).

### SUMMARY OF THE INVENTION

As described below, the present invention features methods of directly targeting specific cell surface receptors on the M2 macrophage for antibody or nanparticle directed therapy.

In a first aspect, the invention features a method of treating or preventing cancer in a subject, comprising administering to a subject having cancer or at risk for cancer an effective amount of one or more binding agents that recognize one or more cell surface markers specific for M2-Tumor Associated Macrophage (TAM), wherein the effective amount of the binding agents is sufficient to treat or prevent the cancer.

In another aspect, the invention features a method of reducing tumor associated macrophage density in a tumor of a subject comprising administering to a subject having a tumor an effective amount of one or more binding agents that recognize one or more cell surface markers specific for M2-Tumor Associated Macrophages (TAMs), wherein the effective amount of the one or more binding agents is sufficient to reduce the density of tumor associated macrophages in the tumor of the subject.

In still another aspect, the invention features a method of staging a tumor in a subject, comprising determining the presence of M2-Tumor Associated Macrophages (TAMs) in the subject.

In another further aspect, the invention features a method of diagnosing or predicting the progression of cancer in a subject, comprising determining the presence of M2 Tumor Associated Macrophages (TAMs) in the subject.

In one embodiment, the determining step comprises contacting a sample of cells from the subject with one or more binding agents that recognize one or more cell surface markers specific for M2-Tumor Associated Macrophage (TAM), and identifying cells recognized by the binding agents.

In another embodiment of the above aspects, the cell surface marker specific for M2-TAM is selected from the group consisting of: CD206 [mannose receptor], IL-4r, IL-1ra, decoy IL-1rII, IL-10r, CD23, macrophage scavenging receptors A and B, Ym-1, Ym-2, Low density receptor-related protein 1 (LRP1), IL-6r, CXCR1/2, CD136, CD14, CD1a, CD1b, CD93, CD226, (FcγR) and PD-L1.

In a further embodiment of the above aspects, the binding agent is coupled to an imaging agent.

In another embodiment of the above aspects, the M2-TAM binding agent is an antibody, or an antigen binding fragment thereof.

In a further embodiment of the above aspects, the antibody is a bispecific antibody, a trispecific antibody, an antibody with greater than three different specificities, or an antigen-binding fragment thereof.

In a related embodiment, the antibody is conjugated to an additional agent. In a further related embodiment, the agent is a toxic agent. In another further embodiment, the toxic agent is a chemotherapeutic drug.

In a further embodiment of the above aspects, the M2-TAM binding agent is a nanoparticle or a liposome. In a related embodiment, the nanoparticle is coated with a M2-TAM cell surface receptor ligand.

In another embodiment of the above aspects, the M2-TAM cell surface receptor ligand is selected from the group consisting of CD206 [mannose receptor], IL-4r, IL-1ra, decoy IL-1rII, IL-10r, CD23, macrophage scavenging receptors A and B, Ym-1, Ym-2, Low density receptor-related protein 1 (LRP1), IL-6r, CXCR1/2, CD136, CD14, CD1a, CD1b, CD93, CD226, (FcγR) and PD-L1.

In one embodiment, the cell surface receptor ligand is coupled to an imaging agent.

In another embodiment, the nanoparticle or liposome comprises an agent. In a related embodiment, the agent is a toxic agent. In another related embodiment, the toxic agent is a chemotherapeutic drug. In another further related embodiment, the toxic agent is a bisphosphonate compound. In still another further embodiment, the toxic agent is a radioactive compound.

In another aspect, the invention features a composition comprising a particle comprising one or more toxic agents and a M2-TAM specific targeting peptide bound to a surface on the particle.

In one embodiment, the particle is a nanoparticle.

In another embodiment of the above aspects, the M2-TAM cell surface receptor ligand is selected from the group consisting of CD206 [mannose receptor], IL-4r, IL-1ra, decoy IL-1rII, IL-10r, CD23, macrophage scavenging receptors A and B, Ym-1, Ym-2, Low density receptor-related protein 1 (LRP1), IL-6r, CXCR1/2, CD136, CD14, CD1a, CD1b, CD93, CD226, (FcγR) and PD-L1.

In another embodiment, the toxic agent is a chemotherapeutic drug. In another related embodiment, the toxic agent is a bisphosphonate compound. In a further related embodiment, the toxic agent is a radioactive compound.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (for instance all exact exemplary values provided with respect to a particular factor or measurement can be considered to also provide a corresponding approximate measurement, modified by “about,” where appropriate).

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the terms “administration” or “administering” are meant to include an act of providing a compound or pharmaceutical composition of the invention to a subject in need of treatment.

As used herein, the term “agent” is meant a polypeptide, polynucleotide, or fragment, or analog thereof, small molecule, or other biologically active molecule.

As used herein, the term “cancer” is meant to refer to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, cancer of the small intestine, and cancer of the esophagus. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. Examples of cancers that are within the scope of the present disclosure include, but are not limited to, carcinoma, breast cancer, ovarian cancer, pancreatic cancer, colon cancer, colorectal cancer, colon cancer, papillary thyroid carcinoma, melanoma, bladder, testicular, head and neck, cervical cancer, lung cancer, Wilms' tumor, brain tumor, neuroblastoma, retinoblastoma, mesothelioma, esophageal cancer or hairy cell leukemia. In particular embodiments, the cancer is melanoma. In some embodiments, the cancer is characterized by increased Ras-BRaf-Mek-Erk signaling, is dependent for growth and/or survival upon the Ras-BRaf-Mek-Erk signaling pathway, and/or expresses an activated or oncogenic BRaf, Ras or Mek. Any mutations in BRaf, Ras and/or Mek are within the scope of the present disclosure. In certain embodiments, the activated or oncogenic BRaf comprises BRafV600E. In other embodiments, the activated or oncogenic Ras comprises RasG12V.

As used herein, the term “cell surface marker specific for M2-TAM” is meant to refer to any cell surface marker expressed on M2-TAM. In certain embodiments, the M2-TAM is selected from the group consisting of CD206 [mannose receptor], IL-4r, IL-1ra, decoy IL-1rII, IL-10r, CD23, macrophage scavenging receptors A and B, Ym-1, Ym-2, Low density receptor-related protein 1 (LRP1), IL-6r, CXCR1/2, CD136, CD14, CD1a, CD1b, CD93, CD226, (FcγR) and PD-L1.

As used herein, the term “chemotherapeutic agent” is meant to refer to agents that are of use in the treatment of cancer.

As used herein, the phrase “in combination with” is intended to refer to all forms of administration that provide the compounds of the invention together, and can include sequential administration, in any order.

As used herein, the term “imaging agent” is meant to refer to a chemical moiety that aids in the visualization of a sample.

As used herein, the term “liposome” is meant to refer to unilamellar or multilamellar vesicles which have a membrane formed from a lipophilic material and an aqueous interior

As used herein, the term “nanoparticle” refers to any particle having a greatest dimension (e.g., diameter) that is less than about 2500 nm. In some embodiments, the dimension is smaller (e.g., less than about 1000 nm, less than about 500 nm less than about 250 nm, less than about 200 nm, less than about 150 nm, less than about 125 nm, less than about 100 nm, less than about 80 nm, less than about 70 nm, less than about 60 nm, less than about 50 nm, less than about 40 nm, less than about 30 nm or even less than about 20 nm). In some embodiments, the dimension is less than about 10 nm. In some embodiments, the nanoparticle is approximately spherical. When the nanoparticle is approximately spherical, the characteristic dimension can correspond to the diameter of the sphere. In addition to spherical shapes, the nanoparticle or other nanoscale material can be disc-shaped, oblong, polyhedral, rod-shaped, cubic, or irregularly-shaped. A nanoscale material can also be irregularly shaped or comprise clusters of spheres, rods, discs, or cubes.

As used herein, the term “M2-Tumor associated macrophage” (TAM) or “alternatively activated macrophage” is meant to refer to a CD206+ macrophage. It is understood that TAMs may be composed of multiple distinct populations with overlapping features that depend on a variety of factors including location in the microenvironment, stage of the tumor, and type of cancer.

As used herein, “predicting the progression” is meant to refer to a determination of the progression of cancer in a subject. Predicting the progression is meant to include a determination of if the cancer will advance or regress in the subject. Predicting the progression can refer to a subject that is being treated with a therapeutic, or overall progression in the presence or absence of therapy.

As used herein, the term “radioactive compound” is meant to refer to any compound that can kill cells through radioactive emission.

As used herein, “staging a tumor” is meant to refer to the process of determining the extent to which a cancer has developed by spreading. Contemporary practice is to assign a number from I-IV to a cancer, with I being an isolated cancer and IV being a cancer which has spread to the limit of what the assessment measures. The stage generally takes into account the size of a tumor, how deeply it has penetrated within the wall of a hollow organ (intestine, urinary bladder), whether it has invaded adjacent organs, how many regional lymph nodes it has metastasized to (if any), and whether it has spread to distant organs.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals (such as sheep, dogs, cats, cows, pigs, etc.), and rodents (such as mice, rats, hamsters, guinea pigs, etc.).

As used herein, the terms “therapeutically effective amount” is meant to refer to an amount of one or more binding agents that recognize one or more cell surface markers specific for M2-Tumor Associated Macrophage (TAM), alone, coupled to another agent, coupled to an imaging agent, or in combination with another agent, that is effective to treat a target disease or condition when administered in combination. In some embodiments, therapeutically effective amount is the amount of each agent in the combination that is sufficient for the combination therapy to be effective in reducing, treating or preventing cancer. The therapeutically effective amount will vary depending upon the specific combination, the subject and disease condition being treated, the weight and age of the subject, the severity of the disease condition, the dosing regimen to be followed, timing of administration, the manner of administration and the like, all of which can be determined readily by one of ordinary skill in the art.

As used herein, the terms “treat,” “treating,” “treatment,” are meant the management and care of a subject, e.g. a mammal, in particular a human, for the purpose of combating the disease, condition, or disorder and includes the administration of the compositions of the present invention to prevent the onset of the symptoms or complications, or alleviating the symptoms or complications, or eliminating the disease, condition, or disorder. It will be appreciated that, although not precluded, treating a disorder or condition does not require that the disorder, condition or symptoms associated therewith be completely eliminated.

### DETAILED DESCRIPTION OF THE INVENTION

The invention features, in part, methods of directly targeting specific cell surface receptors on the M2 macrophage for antibody or nanoparticle directed therapy.

As cellular effectors of the innate immune system, macrophages play essential roles in a myriad of processes, including immune response, inflammation, tissue remodeling, and injury repair (Burke et al. The macrophage. 2nd edition. Oxford; New York: Oxford University Press; 2002.). Macrophages are also major constituents of tumor stroma, and an emerging body of evidence suggests that they play a prominent role in tumor growth and survival. In particular, M2 (alternatively activated) macrophages secrete anti-inflammatory cytokines, promote tissue repair/remodeling, angiogenesis, and elicit downregulation of T-cells and other immune effectors (Lewis CE et al. Cancer Res 2006, 66(2):605-612). Similarly, a number of experimental studies have demonstrated the ability of neoplastic cells to recruit M2 macrophages, which supports tumor growth, stimulates tumor angiogenesis, suppresses host immunity, and promotes invasion and metastasis (Condeelis et al. Cell 2006, 124(2):263-266; Pollard J W. Nat Rev Cancer 2004, 4(1):71-78). Thus, TAMs represent potential targets for novel therapies.

In one aspect, the invention features methods of treating or preventing cancer in a subject, comprising administering to a subject having cancer or at risk for cancer an effective amount of one or more binding agents that recognize one or more cell surface markers specific for M2-Tumor Associated Macrophage (TAM), wherein the effective amount of the binding agents is sufficient to treat or prevent the cancer.

In another aspect, the invention features methods of reducing tumor associated macrophage density in a tumor of a subject comprising administering to a subject having a tumor an effective amount of one or more binding agents that recognize one or more cell surface markers specific for M2-Tumor Associated Macrophages (TAMs), wherein the effective amount of the one or more binding agents is sufficient to reduce the density of tumor associated macrophages in the tumor of the subject.

The presence of M2-Tumor Associted Macrophages can be used to stage a tumor in a subject. Cancer staging is the process of determining the extent to which a cancer has developed by spreading. Staging systems are specific for each type of cancer (e.g., breast cancer and lung cancer). Some cancers, however, do not have a staging system. Although competing staging systems still exist for some types of cancer, the universally-accepted staging system is that of the UICC, which has the same definitions of individual categories as the AJCC.

Accordingly, in another aspect, the invention features methods of staging a tumor in a subject, comprising determining the presence of M2-Tumor Associated Macrophages (TAMs) in the subject.

In another aspect, the invention features methods of diagnosing or predicting cancer in a subject, comprising determining the presence of M2 Tumor Associated Macrophages (TAMs) in the subject.

In one embodiment, the determining step comprises contacting a sample of cells from the subject with one or more binding agents that recognize one or more cell surface markers specific for M2-Tumor Associated Macrophage (TAM), and identifying cells recognized by the binding agents.

Any M2-TAM cell surface receptor ligand is suitable for use in the methods described herein. In certain exemplary embodiments, the M2-TAM cell surface ligand is selected from, but not limited to, CD206 [mannose receptor], IL-4r, IL-1ra, decoy IL-1rII, IL-10r, CD23, macrophage scavenging receptors A and B, Ym-1, Ym-2, Low density receptor-related protein 1 (LRP1), IL-6r, CXCR1/2, CD136, CD14, CD1a, CD1b, CD93, CD226, (FcγR) and PD-L1. 7.

In certain embodiments, the M2-TAM binding agent is an antibody, or an antigen binding fragment thereof.

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The antibody can be a bispecific antibody. A bispecific antibody is an artificial protein that is composed of fragments of two different antibodies and consequently binds to two different types of antigen. The antibody can be a trispecific antibody, an antibody with greater than three different specificities, or an antigen-binding fragment thereof.

An antibody or antibody portion of the invention can be derivatized or linked to another functional molecule (e.g., another peptide or protein). Accordingly, the antibodies and antibody portions of the invention are intended to include derivatized and otherwise modified forms. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding

In certain embodiments, the antibody can be conjugated to an additional agent, as described herein.

In certain embodiments, the M2-TAM binding agent can be a nanoparticle or a liposome. The nanoparticle or liposome preferably comprises an agent, for example a toxic agent, such as a chemotherapeutic drug, a bisphosphonate compound or a radioactive compound.

The nanoparticle can comprise an interior region (i.e., the space between the outer dimensions of the particle) and an outer surface (i.e., the surface that defines the outer dimensions of the particle). In some embodiments, the particle can comprise one or more layers. Thus, for example, a spherical nanoparticle can comprise one or more concentric layers, each successive layer being dispersed over the outer surface of the smaller layer closer to the center of the particle. The particle can be solid or porous or can contain a hollow interior region. In some embodiments, the nanoparticle can comprise two layers, an inner core and an outer layer or shell dispersed over the core.

The term “nanoparticle” refers to any particle having a greatest dimension (e.g., diameter) that is less than about 2500 nm. In some embodiments, the dimension is smaller (e.g., less than about 1000 nm, less than about 500 nm less than about 250 nm, less than about 200 nm, less than about 150 nm, less than about 125 nm, less than about 100 nm, less than about 80 nm, less than about 70 nm, less than about 60 nm, less than about 50 nm, less than about 40 nm, less than about 30 nm or even less than about 20 nm). In some embodiments, the dimension is less than about 10 nm. Agents, such as toxic agents (i.e. chemotherapeutics, bisphosphonate compounds or a radioactive compounds) can be incubated with the nanoparticles, and thereby be associated, embedded, encapsulated, loaded, and/or integrated with nanoparticle.

In some embodiments, nanoparticles comprise a material that is biologically inert and can be physiologically tolerated without significant adverse effects by biological systems. Further, a nanoparticle can be comprised of a biodegradable material. It will be understood that there are no restrictions on the physical parameters of a nanoparticle in embodiments provided herein. The physical parameters of a nanoparticle can be optimized, with the desired effect governing the choice of size and shape.

The nanoparticle can comprise a variety of materials including, but not limited to, polymers such as polystyrene, silicone rubber, polycarbonate, polyurethanes, polypropylenes, polymethylmethacrylate, polyvinyl chloride, polyesters, polyethers, and polyethylene.

Additional examples of polymers include, but are not limited to the following: polyethylene glycol (PEG); poly(lactic acid-co-glycolic acid) (PLGA); copolymers of PLGA and PEG; copolymers of poly(lactide-co-glycolide) and PEG; polyglycolic acid (PGA); copolymers of PGA and PEG; poly-L-lactic acid (PLLA); copolymers of PLLA and PEG; poly-D-lactic acid (PDLA); copolymers of PDLA and PEG; poly-D,L-lactic acid (PDLLA); copolymers of PDLLA and PEG; poly(ortho ester); copolymers of poly(ortho ester) and PEG; poly(caprolactone); copolymers of poly(caprolactone) and PEG; polylysine; copolymers of polylysine and PEG; polyethylene imine; copolymers of polyethylene imine and PEG; polyhydroxyacids; polyanhydrides; polyhydroxyalkanoates, poly(L-lactide-co-L-lysine); poly(serine ester); poly(4-hydroxy-L-proline ester); poly-.alpha.-(4-aminobutyl)-L-glycolic acid; derivatives thereof; combinations thereof; and copolymers thereof.

Additional examples of polymeric and non-polymeric materials that can be used is several embodiments include, but are not limited to, poly(lactide), poly(hydroxybutyrate), poly(beta-amino) esters and/or copolymers thereof. Alternatively, the particles can comprise other materials, including but not limited to, poly(dienes) such as poly(butadiene) and the like; poly(alkenes) such as polyethylene, polypropylene and the like; poly(acrylics) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinyl halides) such as poly(vinyl chloride) and the like; poly(vinyl nitriles), poly(vinyl esters) such as poly(vinyl acetate) and the like; poly(vinyl pyridines) such as poly(2-vinyl pyridine), poly(5-methyl-2-vinyl pyridine) and the like; poly(styrenes); poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose and the like; cellulose esters such as cellulose acetate, cellulose acetate phthalate, cellulose acetate butyrate, and the like; poly(saccharides), protein, polypeptides, gelatin, starch, gums, resins and the like. These materials may be used alone, as physical mixtures (blends), or as copolymers.

Biodegradable, biopolymer (e.g. polypeptides such as BSA, polysaccharides, etc.), other biological materials (e.g. carbohydrates), and/or polymeric compounds are also suitable for use as a nanoparticle scaffold. In various embodiments, the nanoparticle is negatively charged. The nanoparticles may themselves have a negative charge or alternatively a positive charge on them or may be modified to attach a negative charge or positive charge to the scaffold, such as, but not limited to, aldehyde, amine, carboxyl, sulfate, or hydroxyl groups. Factors such as nanoparticle surface charge and hydrophilic/hydrophobic balance of these polymeric materials can be achieved by synthetic modification of the polymers. Such synthetic modification is known in the art and contemplated herein. Various methods for producing the negatively charged nanoparticles are described in U.S. Pat. No. 7,390,384, which is incorporated herein by reference in its entirety.

Liposomes can be used as effective drug delivery vehicles, and commercially available liposomal products have been developed for treatment of diseases including cancer (Barenholz, Y., Curr. Opin. in Colloid & Interface Sci. 6(1): 66-77 (2001)). A liposome is a vesicle including at least one phospholipid bilayer separating an interior aqueous phase from the external aqueous environment. A liposome is capable of carrying both hydrophobic cargo in the lipid bilayer and/or hydrophilic cargo in the aqueous core.

Liposomes are unilamellar or multilamellar vesicles which have a membrane formed from a lipophilic material and an aqueous interior. The aqueous interior portion contains the composition to be delivered. Phospholipids used for liposome formation include, but are not limited to, natural phospholipids such as egg yolk lecithin (phosphatidyl choline), soybean lecithin, lysolecithin, sphingomyelin, phosphatidic acid, phosphatidyl serine, phosphatidyl glycerol, phosphatidyl inositol, phosphatidyl ethanolamine, diphosphatidyl glycerol. Liposome preparation is described, for example, in U.S. Pat. Nos. 7,208,174, 7,108,863, 5,192,549, 6,958,241, and in Ann Rev. Biophys. Bioeng., 9, 467 (1980), “Liposomes” (Ed. by M. J. Ostro, Marcel Dekker, Inc.) the entire contents of which are incorporated herein by reference. In several embodiments, one or more DNA repair enzyme(s), whether present as a component of an extract or in isolated or purified form, are contained in multilamellar liposomes.

When phospholipids and many other amphipathic lipids are dispersed gently in an aqueous medium they swell, hydrate and spontaneously form multilamellar concentric bilayer vesicles with layers of aqueous media separating the lipid bilayers. These systems commonly are referred to as multilamellar liposomes or multilamellar vesicles (MLV) and usually have diameters of from 0.2 um to 5 um. Sonication of MLV results in the formation of small unilamellar vesicles (SUV) with diameters usually in the range of 20 to 100 nm, containing an aqueous solution in the core. Multivesicular liposomes (MVL) differ from multilamellar liposomes in the random, non-concentric arrangement of chambers within the liposome. Amphipathic lipids can form a variety of structures other than liposomes when dispersed in water, depending on the molar ratio of lipid to water, but at low ratios the liposome is the preferred structure.

The physical characteristics of liposomes generally depend on pH and ionic strength. They characteristically show low permeability to ionic and polar substances, but at certain temperatures can undergo a gel-liquid crystalline phase (or main phase) transition dependent upon the physical properties of the lipids used in their manufacture which markedly alters their permeability. The phase transition involves a change from a closely packed, ordered structure, known as the gel state, to a loosely packed, less-ordered structure, known as the liquid crystalline state.

Various types of lipids differing in chain length, saturation, and head group have been used in liposomal formulations for years, including the unilamellar, multilamellar, and multivesicular liposomes mentioned above.

There are at least three types of liposomes. The term “multivesicular liposomes (MVL)” generally refers to man-made, microscopic lipid vesicles comprising lipid membranes enclosing multiple non-concentric aqueous chambers. In contrast, “multilamellar liposomes or vesicles (MLV)” have multiple “onion-skin” concentric membranes, in between which are shell-like concentric aqueous compartments. Multilamellar liposomes and multivesicular liposomes characteristically have mean diameters in the micrometer range, usually from 0.5 to 25 um. The term “unilamellar liposomes or vesicles (ULV)” generally refers to liposomal structures having a single aqueous chamber, usually with a mean diameter range from about 20 to 500 nm.

Multilamellar and unilamellar liposomes can be made by several relatively simple methods. A number of techniques for producing ULV and MLV are described in the art (for example in U.S. Pat. No. 4,522,803 to Lenk; U.S. Pat. No. 4,310,506 to Baldeschweiler; U.S. Pat. No. 4,235,871 to Papahadjopoulos; U.S. Pat. No. 4,224,179 to Schneider, U.S. Pat. No. 4,078,052 to Papahadjopoulos; U.S. Pat. No. 4,394,372 to Taylor U.S. Pat. No. 4,308,166 to Marchetti; U.S. Pat. No. 4,485,054 to Mezei; and U.S. Pat. No. 4,508,703 to Redziniak).

By contrast, production of multivesicular liposomes generally requires several process steps. Briefly, a common method for making MVL is as follows: The first step is making a “water-in-oil” emulsion by dissolving at least one amphipathic lipid and at least one neutral lipid in one or more volatile organic solvents for the lipid component, adding to the lipid component an immiscible first aqueous component and a biologically active substance to be encapsulated, and optionally adding, to either or both the lipid component and the first aqueous component, an acid or other excipient for modulating the release rate of the encapsulated biologically active substances from the MVL. The mixture is emulsified, and then mixed with a second-immiscible aqueous component to form a second emulsion. The second emulsion is mixed either mechanically, by ultrasonic energy, nozzle atomization, and the like, or by combinations thereof, to form solvent spherules suspended in the second aqueous component. The solvent spherules contain multiple aqueous droplets with the substance to be encapsulated dissolved in them (see Kim et al., Biochem. Biophys. Acta, 728:339-348, 1983). For a comprehensive review of various methods of ULV and MLV preparation, refer to Szoka, et al. Ann. Rev. Biophys. Bioeng. 9:465-508, 1980.

Making multivesicular liposomes can involve inclusion of at least one amphipathic lipid and one neutral lipid in the lipid component. The amphipathic lipids can be zwitterionic, anionic, or cationic lipids. Examples of zwitterionic amphipathic lipids are phosphatidylcholines, phosphatidylethanolamines, sphingomyelins etc. Examples of anionic amphipathic lipids are phosphatidylglycerols, phosphatidylserines, phosphatidylinositols, phosphatidic acids, etc. Examples of cationic amphipathic lipids are diacyl trimethylammoniumpropane and ethyl phosphatidylcholine. Examples of neutral lipids include diglycerides, such as diolein, dipalmitolein, and mixed caprylin-caprin diglycerides; triglycerides, such as triolein, tripalmitolein, trilinolein, tricaprylin, and trilaurin; vegetable oils, such as soybean oil; animal fats, such as lard and beef fat; squalene; tocopherol; and combinations thereof. Additionally, cholesterol or plant sterols can be used in making multivesicular liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

In exemplary embodiments described herein, liposomes that contain one or more agents, can be of various compositions. For example, the liposomes may be made from natural and synthetic phospholipids, glycolipids, and other lipids and lipid congeners; cholesterol, cholesterol derivatives and other cholesterol congeners; charged species which impart a net charge to the membrane; reactive species which can react after liposome formation to link additional molecules to the liposome membrane; and other lipid soluble compounds which have chemical or biological activity.

Liposomes can be composed of phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions can be formed from dimyristoyl phosphatidylglycerol, while anionic fusogenic liposomes can be formed from dioleoyl phosphatidylethanolamine (DOPE). Another type of liposomal composition can be formed from phosphatidylcholine (PC) such as, for example, soybean PC, and egg PC. Another type can be formed from mixtures of phospholipid and/or phosphatidylcholine and/or cholesterol.

In exemplary embodiments, the nanoparticle or liposome is coated with a M2-TAM cell surface receptor ligand.

### Additional Agents

In certain embodiments of the invention, the M2-TAM binding agent is conjugated or coupled to an additional agent. The additional agent can be, for example, a toxic agent. A “toxic agent” is meant to refer to any agent that can kill a cell. The toxic agent can be a chemotherapeutic drug, a bisphosphonate compound or a radioactive compound, but is not meant to be limited as such.

Examples of chemotherapeutic agents are described in the scientific and patent literature and can be readily determined by those skilled in the art (see, e.g., Bulinski, J. C. et al. (1997) J. Cell Sci. 110:3055-3064; Panda, D. et al. (1997) Proc. Natl. Acad. Sci. USA 94: 10560-10564; Muhlradt, P. F. et al. (1997) Cancer Res. 57:3344-3346; Nicolaou, K. C. et al. (1997) Nature 387:268-272; Vasquez, R. J. et al. (1997) Mol. Biol. Cell. 8:973-985; Panda, D. et al. (1996) J. Biol. Chem. 271 :29807-29812). Examples of some classes of chemotherapeutic and anti-cancer agents include, but are not limited to, the following: alkylating agents, anti-EGFR antibodies, anti-Her-2 antibodies, antimetabolites, vinca alkaloids, anthracyclines, topoisomerases, taxanes, epothilones, antibiotics, immunomodulators, immune cell antibodies, interferons, interleukins, HSP90 inhibitors, anti-androgens, antiestrogens, anti-hypercalcaemia agents, apoptosis inducers, Aurora kinase inhibitors, Bruton's tyrosine kinase inhibitors, calcineurin inhibitors, CaM kinase II inhibitors, CD45 tyrosine phosphatase inhibitors, CDC25 phosphatase inhibitors, cyclooxygenase inhibitors, cRAF kinase inhibitors, cyclin dependent kinase inhibitors, cysteine protease inhibitors, DNA intercalators, DNA strand breakers, E3 ligase inhibitors, EGF pathway inhibitors, farnesyltransferase inhibitors, Flk-1 kinase inhibitors, glycogen synthase kinase-3 inhibitors, histone deacetylase inhibitors, I-kappa B-alpha kinase inhibitors, imidazotetrazinones, insulin tyrosine kinase inhibitors, c-Jun-N-terminal kinase inhibitors, mitogen-activated protein kinase inhibitors, MDM2 inhibitors, MEK inhibitors, MMP inhibitors, mTor inhibitors, NGFR tyrosine kinase inhibitors, p38 MAP kinase inhibitors, p56 tyrosine kinase inhibitors, PDGF pathway inhibitors, phosphatidylinositol-3-kinase inhibitors, phosphatase inhibitors, protein phosphatase inhibitors, PKC inhibitors, PKC delta kinase inhibitors, polyamine synthesis inhibitors, proteasome inhibitors, PTP1B inhibitors, SRC family tyrosine kinase inhibitors, Syk tyrosine kinase inhibitors, Janus (JAK-2 and/or JAK-3) tyrosine kinase inhibitors, retinoids, RNA polymerase II elongation inhibitors, Serine/Threonine kinase inhibitors, sterol biosynthesis inhibitors, VEGF pathway inhibitors, immunosuppressive agents, CYP3A4 inhibitors, anti-microbial agents, and antiemetics.

The term “bisphosphonate compound” includes all forms thereof including stereoisomers, enantiomers, diastereomers, racemic mixtures and derivatives thereof, for example, salts, acids, esters and the like. Bisphosphonate compounds are synthetic analogues of pyrophosphate (structure P—O—P) in which the central oxygen atom is replaced with a carbon atom. Established nomenclature in the art categorizes bisphosphonates into three generations. The first category comprises the “first-generation” compounds which do not contain a nitrogen atom in their side chains R¹ and R². This category includes, for example, etidronate, clodronate and tiludronate. The secondary category includes the “second-generation” and “third-generation” compounds which contain one or more nitrogen atoms in one of their side chains R¹ or R². Those of the second generation comprise an aliphatic side chain bearing a nitrogen atom or a terminal NH₂ group. Examples include pamidronate, alendronate, ibandronate and neridronate. Those of the third generation bear a heterocyclic nucleus containing a nitrogen atom. Examples include risedronate and zoledronate (imidazole nucleus).

Non-limiting examples of bisphosphonates useful herein include the following: 1-hydroxy-2-(3-pyridinyl)-ethylidene-1,1-bisphosphonic acid (risedronate) as described in U.S. Pat. No. 5,583,122, to Benedict et al., issued Dec. 10, 1996; U.S. Pat. No. 6,410,520 B2, to Cazer et al., issued Jun. 25, 2002; 4-amino-1-hydroxybutylidene-1,1-bisphosphonic acid (alendronic acid or alendronate) as described in U.S. Pat. No. 4,621,077, to Rosini et al., issued Nov. 4, 1986; U.S. Pat. No. 6,281,381 B1, to Finkelstein et al., issued Aug. 28, 2001; U.S. Pat. No. 6,008,207, to Brenner et al., issued Dec. 28, 1999; U.S. Pat. No. 5,849,726, to Brenner et al., issued Dec. 15, 1998; U.S. Pat. Pub. 2001/0021705 A1, by Brenner et al., published Sep. 13, 2001; U.S. Pat. No. 4,922,007, to Kieczykowski et al., issued May 1, 1990; U.S. Pat No. 5,019,651, to Kieczykowski, issued May 28, 1991; 3-amino-1-hydroxypropylidene-1,1-bisphosphonic acid (pamidronate) as described in U.S. Pat. No. 4,639,338, to Stahl et al., issued Jan. 27, 1987; (4-chlorophenyl)thiomethane-1,1-diphosphonic acid (tiludronate) as described in U.S. Pat. No. 4,876,248 to Breliere et al., issued Oct. 24, 1989; 1,1-dichloromethylene-1,1-diphosphonic acid (clodronate) as described in U.S. Pat. No. 3,422,021; cycloheptylaminomethylene-1,1-bisphosphonic acid (cimadronate), as described in U.S. Pat. No. 4,970,335, to Isomura et al., issued Nov. 13, 1990; 1-hydroxy-3-(N-methyl-N-pentylamino)propylidene-1,1-bisphosphonic acid (ibandronate), which is described in U.S. Pat. No. 4,927,814, issued May 22, 1990; 1-hydroxy-2-(imidazol-1-yl)ethane-1,1-bisphosphonic acid (zoledronate); and 1-(N-phenylaminothiocarbonyl)methane-1,1-bisphosphonic acid.

In some embodiments, the bisphosphonate compound is selected from the group consisting of risedronate, alendronate, pamidronate, tiludronate, cimadronate, ibandronate, clodronate, zoledronate, and salts, esters, hydrates, hemihydrates, polymorphs, and solvates thereof, and combinations thereof.

Additional non-limiting examples of bisphosphonate compounds are disclosed in U.S. Patent Application No. 2010/0316676, which is herein incorporated by reference in its entirety.

A radioactive compound refers to any compound that can kill cells by radioactive emission. Rapidly dividing cells are particularly sensitive to damage by radiation. Internal radiotherapy is by administering or planting a small radiation source, usually a gamma or beta emitter, in the target area.

Radioactive agents may include, but are not limited to, Fibrinogen 1 125; Fludeoxyglucose F18; Fluorodopa F 18; Insulin I 125; Insulin I 131; Iobenguane I 123; Iodipamide Sodium I 131; Iodoantipyrine 1 131; Iodocholesterol 1 131; Iodohippurate Sodium 1 123; Iodohippurate-23-Sodium 1 125; Iodohippurate Sodium 1 131;

Iodopyracet I 125; Iodopyracet I 131; Iofetamine Hydrochloride I 123; Iomethin 1 125; Iomethin 1 131; Iothalamate Sodium 1 125; Iothalamate Sodium 1 131; Iotyrosine 1 131; Liothyronine I 125; Liothyronine 1 131; Merisoprol Acetate Hg 197; Merisoprol Acetate-Hg 203; Merisoprol Hg 197; Selenomethionine Se 75; Technetium Tc 99m Atimony Trisulfide Colloid; Technetium Tc 99m Bicisate; Technetium Tc 99m Disofenin;

Technetium Tc 99m Etidronate; Technetium Tc 99m Exametazime; Technetium Tc 99m Furifosmin; Technetium Tc 99m Gluceptate; Technetium 99m Lidofenin; Technetium Tc 99mm Mebrofenin; Technetium Tc 99m Medronate; TechnetiumTc 99m Medronate Disodium; Technetium Tc 99m Mertiatide; Technetium Tc 99m Oxidronate; Technetium Tc 99m Pentetate; Technetium Ic 99m Pentetate Calcium Trisodium; Technetium Tc 99m Sestamibi; Technetium Tc 99m Siboroxime; Technetium Tc 99m Succimer; Technetium Tc 99m Sulfur Colloid; Technetium Tc 99m Teboroxime; Technetium Tc 99m Tetrofosmin; Technetium Tc 99m Tiatide; Thyroxine 1 125: Thyroxine 1 131;

Tolpovidone 1 131; Triolein 1 125; Triolein I 131.

The cell surface receptor ligand may be coupled to an imaging agent. The term “imaging agent” is meant to refer to any chemical moiety that aids in the visualization of a sample.

For example, imaging agents that are detectable using X-ray technologies (e.g., X-rays, CT/CAT scans) and magnetic resonance imaging (MRI) are well known and widely used in the medical diagnostics field. Broadly speaking, the agents possess a property that can be detected by a particular detection device. When introduced into the body of a patient (used interchangeably herein with “subject” and “animal”), the presence of the agent at a site of interest (e.g., a target tissue) allows an image of the site to be created, thus allowing the medical practitioner to view and assess the site. Use of such agents is possible in numerous diseases and disorders, and for a wide range of tissues and organs in animals.

An imaging agent can be a “contrast agent”, and can refer to a moiety (a specific part of or an entire molecule, macromolecule, coordination complex, or nanoparticle) that increases the contrast of a biological tissue or structure being examined. The contrast agent can increase the contrast of a structure being examined using magnetic resonance imaging (MRI), optical imaging, positron emission tomography (PET) imaging, single photon emission computed tomography (SPECT) imaging, or a combination thereof (i.e., the contrast agent can be multimodal).

An “optical imaging agent” or “optical contrast agent” refers to a group that can be detected based upon an ability to absorb, reflect or emit light (e.g., ultraviolet, visible, or infrared light). Optical imaging agents can be detected based on a change in amount of absorbance, reflectance, or fluorescence, or a change in the number of absorbance peaks or their wavelength maxima. Thus, optical imaging agents include those which can be detected based on fluorescence or luminescence, including organic and inorganic dyes.

A “MRI contrast agent” or “MRI imaging agent” refers to a moiety that effects a change in induced relaxation rates of water protons in a sample. MRI contrast agents typically employ paramagnetic metal ions to effect such changes.

A “fluorophore” refers to a species that can be excited by visible light or non-visible light (e.g., UV light). Examples of fluorophores include, but are not limited to: quantum dots and doped quantum dots (e.g., a semiconducting CdSe quantum dot or a Mn-doped CdSe quantum dot), fluorescein, fluorescein derivatives and analogues, indocyanine green, rhodamine, triphenylmethines, polymethines, cyanines, phalocyanines, naphthocyanines, merocyanines, lanthanide complexes or cryptates, fullerenes, oxatellurazoles, LaJolla blue, porphyrins and porphyrin analogues and natural chromophores/fluorophores such as chlorophyll, carotenoids, flavonoids, bilins, phytochrome, phycobilins, phycoerythrin, phycocyanines, retinoic acid and analogues such as retinoins and retinates.

### Pharmaceutical Compositions and Administration

The invention features a composition comprising a particle comprising one or more toxic agents and a M2-TAM specific targeting peptide bound to a surface on the particle. The particles comprising one or more toxic agents and a M2-TAM specific targeting peptide bound to a surface on the particle can be administered in a variety of ways and pharmaceutical forms in the embodiments provided herein for reducing tumor associated macrophage density or treating or preventing cancer. As such, provided herein are several compositions drawn to pharmaceutical compositions comprising the particles as described herein and a pharmaceutically acceptable carrier or diluent depending on the route and form of administration.

As used herein, the term “particle” refers to a delivery, i.e. a drug delivery vehicle, vehicle not limited to any size, shape, or dimension, and having a surface to which a tumor specific targeting peptide can be attached and capable of delivering an agent, such as a toxic agent. In some aspects, the particles can include, but is not limited to nanospheres, nanoparticles, microcapsules, nanocapsules, microspheres, microparticles, colloids, aggregates, flocculates, insoluble salts, emulsions and insoluble complexes, any of which can comprise inorganic materials, polymers, polypeptides, proteins, lipids, and surfactants.

In one embodiment, the particle is a nanoparticle. In another embodiment, the M2-TAM specific targeting peptide is selected from the group consisting of: CD206 [mannose receptor], IL-4r, IL-1ra, decoy IL-1rII, IL-10r, CD23, macrophage scavenging receptors A and B, Ym-1, Ym-2, Low density receptor-related protein 1 (LRP1), IL-6r, CXCR1/2, CD136, CD14, CD1a, CD1b, CD93, CD226, (FcγR) and PD-L1.

Examples of routes of administration that may be used include injection (subcutaneous, intravenous, parenterally, intraperitoneally, intrathecal), or oral routes. The pharmaceutical preparations may be given by forms suitable for each administration route. For example, these preparations can be administered in tablets or capsule form, by injection or orally. The injection can be bolus or can be continuous infusion. The particles comprising one or more toxic agents and a M2-TAM specific targeting peptide bound to a surface on the particle can be administered alone, or in conjunction with either another agent or agents known in the art for treating cancer or with a pharmaceutically-acceptable carrier,or both.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, polyethylene glycol (PEG).

The compositions may be in the “pharmaceutical form” of tablets, capsules, powders, granules, lozenges, liquid or gel preparations. Tablets and capsules for oral administration may be in a form suitable for unit dose presentation and may contain conventional excipients. Examples of these are: binding agents such as syrup, acacia, gelatin, sorbitol, tragacanth, and polyvinylpyrrolidone; fillers such as lactose, sugar, maize-starch, calcium phosphate, sorbitol or glycine; tableting lubricants, such as magnesium stearate, silicon dioxide, talc, polyethylene glycol or silica; disintegrants, such as potato starch; or acceptable wetting agents, such as sodium lauryl sulfate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, e.g., sorbitol, syrup, methyl cellulose, glucose syrup, gelatin, hydrogenated edible fats, emulsifying agents, e.g., lecithin, sorbitan monooleate, or acacia; non-aqueous vehicles (including edible oils), e.g., almond oil, fractionated coconut oil, oily esters such as glycerine, propylene glycol, or ethyl alcohol; preservatives such as methyl or propyl p-hydroxybenzoate or sorbic acid, and, if desired, conventional flavoring or coloring agents.

Solid dosage forms for oral administration include capsules, tablets, pills, powders, and granules. In such solid dosage forms, the particles comprising one or more toxic agents and a M2-TAM specific targeting peptide bound to a surface on the particle can be admixed with at least one inert pharmaceutically acceptable carrier such as sucrose, lactose, or starch. Such dosage forms can also comprise, as is normal practice, additional substances other than inert diluents, for example, lubricating agents such as magnesium stearate. In the case of capsules, tablets, and pills, the dosage forms may also comprise buffering agents. Tablets and pills can additionally be prepared with enteric coatings known in the art.

Liquid dosage forms for oral administration include pharmaceutically acceptable emulsions, solutions, suspensions, syrups, with the elixirs containing inert diluents commonly used in the art, such as water. Besides such inert diluents, compositions can also include adjuvants, such as wetting agents, emulsifying and suspending agents, and sweetening, flavoring, and perfuming agents.

The particles comprising one or more toxic agents and a M2-TAM specific targeting peptide bound to a surface on the particle can also be administered parenterally. The phrases “parenteral administration” and “administered parenterally” as used herein includes, for example, modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. Parenteral administration can include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

For parenteral administration, the peptides can be, for example, formulated as a solution, suspension, emulsion or lyophilized powder in association with a pharmaceutically acceptable parenteral vehicle. Examples of such vehicles are water, saline, Ringer's solution, dextrose solution, and 5% human serum albumin Liposomes and nonaqueous vehicles such as fixed oils may also be used. The vehicle or lyophilized powder may contain additives that maintain isotonicity (for example, sodium chloride, mannitol) and chemical stability (for example, buffers and preservatives). The formulation is sterilized by commonly used techniques. For example, a parenteral composition suitable for administration by injection is prepared by dissolving 1.5% by weight of active ingredient in 0.9% sodium chloride solution.

The pharmaceutical compositions described herein can be administered as a single dose or in multiple doses; administered either as individual therapeutic agents or in combination with other therapeutic agents; and combined with conventional therapies, which may be administered sequentially or simultaneously.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

Alternatively activated macrophages, M2-TAMs, are an abundant part of solid and hematological malignancies and have been linked with progression, metastasis and resistance to therapy (22,23). Strategies for inhibiting M2-TAMs are classically grouped into four categories: (i) inhibiting macrophage recruitment; (ii) suppressing TAM survival; (iii) enhancing M1-like tumoricidal activity of TAMs; (iv) blocking M2-like tumor-promoting activity of TAMs (22,23). The present experiments are directed to blocking M2-TAM tumor promotion by identifying surface antigens/combinations of antigens on the M2-TAM for antibody directed therapy or for nanoparticle directed therapy.

### Example 1. Characterize the Sensitivity and Specificity of Known M2 Cell Surface Antigens

In one set of experiments, the sensitivity and specificity of known M2 cell surface antigens will be characterized. Examples of known M2 cell surface antigens include, but are not limited to, CD206 [mannose receptor], IL-4r, IL-1ra, decoy IL-1rII, IL-10r, CD23, macrophage scavenging receptors A and B, Ym-1, Ym-2, Low density receptor-related protein 1 (LRP1), IL-6r, CXCR1/2, CD136, CD14, CD1a, CD1b, CD93, CD226, (FcγR) and PD-L1. Antibody-drug conjugates are generated to single antigens or combinations of antigens (e.g., bispecific antibodies) for M2-TAM targeting.

### Example 2. Coated Nanoparticles

In another set of experiments, a nanoparticle is coated with mannose to allow binding to the M2-TAM. This nanoparticle can then be loaded with a toxic agent to result in M2-TAM destruction (e.g., [but not limited to] bisphosphonates).

### Example 3. Identification of Cell Surface Targets on M2-TAMs

In another set of experiments, novel cell surface targets on M2-TAMs as compared to other macrophage types and monocytes will be identified through discovery of differential characterization of cell surface markers.

This analysis will be done with samples from healthy volunteers as well as patients with cancer that are differentiated to the M1 versus M2 phenotypes. Antibody-drug conjugates are generated to single antigens or combinations of antigens (e.g., bispecific antibodies) for M2-TAM targeting.

### Example 4. M2-TAM Targets

In another set of experiments, universal as well as cancer specific M2-TAM targets are identified. Gene expression and proteomic patterns will be discerned for M2-TAMs from different cancers to determine if targets are cancer-type specific or are generalizable across tumor types. These experiments will be first done utilizing human prostate, breast, lung, and pancreatic tumors in mice. Expansion to other tumor types will be as needed. Differential characterization of cell surface markers utilizing monocytes from healthy volunteers as well as patients with cancer that are differentiated to the M1 versus M2 phenotypes as needed.

### Example 5. Differentiation of Monocytes to M2 Versus M1 TAMs

Cell surface antigens may change depending on what molecules are utilized to push differentiation of monocytes to M2 versus M1 TAMs. Traditionally, IL-4 and IL-13 are utilized. Different combinations of cytokines can be utilized to determine the optimal strategy for educating monocytes to differentiate to M2-TAMs.

### Example 6. Targeting M2-TAMs for Cancer Therapy

In this example, an antibody, e.g, CD206, is conjugated to antimitotic agent, monomethyl auristatin E to directly kill the M2-TAMs.

### Example 7. Characterize the Sensitivity and Specificity of Known M2 cell Surface Antigens for Imaging

In one set of experiments, the sensitivity and specificity of known M2 cell surface antigens will be characterized. Examples of known M2 cell surface antigens include, but are not limited to, CD206 [mannose receptor], IL-4r, IL-1ra, decoy IL-1rII, IL-10r, CD23, macrophage scavenging receptors A and B, Ym-1, Ym-2, Low density receptor-related protein 1 (LRP1), IL-6r, CXCR1/2, CD136, CD14, CD1a, CD1b, CD93, CD226, (FcγR) and PD-L1. Antibody-imaging agent conjugates are generated to single antigens or combinations of antigens (e.g., bispecific antibodies) for M2-TAM imaging, allowing for identication of tumor masses.

### Example 8. Characterize the Sensitivity and Specificity of M2 Cell Surface Antigens for Diagnostic Studies

In one set of experiments, the sensitivity and specificity of M2 cell surface antigens will be characterized and patterned. M2 cell surface antigens include those that are known, for example CD206 [mannose receptor], IL-4r, IL-1ra, decoy IL-1rII, IL-10r, CD23, macrophage scavenging receptors A and B, Ym-1, Ym-2, Low density receptor-related protein 1 (LRP1), IL-6r, CXCR1/2, CD136, CD14, CD1a, CD1b, CD93, CD226, (FcγR) and PD-L1. However, it is understood that new M2 cell surface antigens may be discovered, and are meant to be included among the M2 cell surface antigens. Patterns of receptors in patients will be characterized to diagnose cancer and/or cancer types in patients.

### Example 9. Characterize the Sensitivity and Specificity of M2 Cell Surface Antigens for Diagnostic Studies

In one set of experiments, the sensitivity and specificity of M2 cell surface antigens will be characterized and patterned. M2 cell surface antigens include those that are known, for example CD206 [mannose receptor], IL-4r, IL-1ra, decoy IL-1rII, IL-10r, CD23, macrophage scavenging receptors A and B, Ym-1, Ym-2, Low density receptor-related protein 1 (LRP1), IL-6r, CXCR1/2, CD136, CD14, CD1a, CD1b, CD93, CD226, (FcγR) and PD-L1. However, it is understood that new M2 cell surface antigens may be discovered, and are meant to be included among the M2 cell surface antigens. Patterns of receptors in patients will be characterized to estimate prognosis in cancer and/or cancer types in patients.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

1. Li X, Loberg R, Liao J, Ying C, Snyder L A, Pienta K J, McCauley L K. A destructive cascade mediated by CCL2 facilitates PCa growth in bone. Cancer Res. 2009 Feb. 15; 69(4):1685-92.

2. Lewis C E, Pollard J W. Distinct role of macrophages in different tumor microenvironments. Cancer Res, 66: 605-612, 2006.

3. Mantovani A, Bottazzi B, Colotta F, Sozzani S, Ruco L. The origin and function of tumor-associated macrophages. Immunol Today, 13: 265-270, 1992.

4. Sica A, Schioppa T, Mantovani A, and Allavena P. Tumour-associated macrophages are a distinct M2 polarised population promoting tumour progression: potential targets of anti-cancer therapy. Eur J Cancer, 42: 717-727, 2006.

5. Zhang J, Lu Y, Pienta K J. Multiple Roles of Chemokine (C—C Motif) Ligand 2 in Promoting PCa Growth. J Natl Cancer Inst. 2010 Mar. 30. PMCID: 2857800

6. Roca H, Varsos Z S, Sud S, Craig M J, Ying C, Pienta K J. CCL2 and interleukin-6 promote survival of human CD11b+ peripheral blood mononuclear cells and induce M2-type macrophage polarization. J Biol Chem. 2009 Dec. 4; 284(49):34342-54.

7. Loberg R D, Day L L, Harwood J, Ying C, St John L N, Giles R, Neeley C K, Pienta K J. CCL2 is a potent regulator of PCa cell migration and proliferation. Neoplasia, 8: 578-586, 2006. PMCID: PMC1601934

8. Kelly P M, Davison R S, Bliss E, McGee J O. Macrophages in human breast disease: a quantitative immunohistochemical study. Br J Cancer, 57: 174-177, 1988.

9. Crowther M, Brown N J, Bishop, E T, Lewis C E. Microenvironmental influence on macrophage regulation of angiogenesis in wounds and malignant tumors. J Leukoc Biol, 70: 478-490, 2001.

10. Stephens T C, Currie G A, Peacock, J H. Repopulation of gamma-irradiated Lewis lung carcinoma by malignant cells and host macrophage progenitors. Br J Cancer, 38: 573-582, 1978.

11. Murdoch C, Giannoudis A, Lewis C E. Mechanisms regulating the recruitment of macrophages into hypoxic areas of tumors and other ischemic tissues. Blood, 104: 2224-2234, 2004.

12. Milliken, D., Scotton, C., Raju, S., Balkwill, F., and Wilson, J. Analysis of chemokines and chemokine receptor expression in ovarian cancer ascites. Clin Cancer Res, 8: 1108-1114, 2002.

13. Azenshtein, E., Luboshits, G., Shina, S., Neumark, E., Shahbazian, D., Weil, M., Wigler, N., Keydar, I., and Ben-Baruch, A. The CC chemokine RANTES in breast carcinoma progression: regulation of expression and potential mechanisms of promalignant activity. Cancer Res, 62: 1093-1102, 2002.

14. Lissbrant, I. F., Stattin, P., Wikstrom, P., Damber, J. E., Egevad, L., and Bergh, A. Tumor associated macrophages in human PCa: relation to clinicopathological variables and survival. Int J Oncol, 17: 445-451, 2000.

15. Zollo M, Di Dato V, Spano D, De Martino D, Liguori L, Marino N, Vastolo V, Navas L, Garrone B, Mangano G, Biondi G, Guglielmotti A. Targeting monocyte chemotactic protein-1 synthesis with bindarit induces tumor regression in prostate and breast cancer animal models. Clin Exp Metastasis. 2012 August;29(6):585-601.

16. Sameni M, Anbalagan A, Olive M B, Moin K, Mattingly R R, Sloane B F. MAME models for 4D live-cell imaging of tumor: microenvironment interactions that impact malignant progression. J Vis Exp. 2012 Feb. 17; (60).

17. Vykhovanets E V, Maclennan G T, Vykhovanets O V, Gupta S. IL-17 Expression by macrophages is associated with proliferative inflammatory atrophy lesions in PCa patients. Int J Clin Exp Pathol. 2011 Aug. 15; 4(6):552-65

18. Sroka I C, Sandoval C P, Chopra H, Gard J M, Pawar S C, Cress A E. Macrophage-dependent cleavage of the laminin receptor α6β1 in PCa. Mol Cancer Res. 2011 October; 9(10):1319-28

19. Roca H, Hernandez J, Weidner S, McEachin R C, Fuller D, Sud S, Schumann T, Wilkinson J E, Zaslaysky A, Li H, Maher C A, Daignault-Newton S, Healy P N, Pienta K J. Transcription factors OVOL1 and OVOL2 induce the mesenchymal to epithelial transition in human cancer. PLOS1, In Press, 2013.

20. Loberg R D, Ying C, Craig M, Day L L, Sargent E, Neeley C, Wojno K, Snyder L A, Yan L, Pienta K J. Targeting CCL2 with systemic delivery of neutralizing antibodies induces prostate cancer tumor regression in vivo. Cancer Res. 2007 Oct. 1; 67(19):9417-24.

21. Pienta K J, Machiels J P, Schrijvers D, Alekseev B, Shkolnik M, Crabb S J, Li S, Seetharam S, Puchalski T A, Takimoto C, Elsayed Y, Dawkins F, de Bono J S. Phase 2 study of carlumab (CNTO 888), a human monoclonal antibody against CC-chemokine ligand 2 (CCL2), in metastatic castration-resistant PCa. Invest New Drugs. 2013 June; 31(3):760-8.

22. Cook J, Hagemann T. Tumour-associated macrophages and cancer. Curr Opin Pharmacol. 2013 August; 13(4):595-601

23. Tang X, Mo C, Wang Y, Wei D, Xiao H. Anti-tumour strategies aiming to target tumour-associated macrophages. Immunology. 2013 February; 138(2):93-104.

What is claimed is:

1. A method of treating or preventing cancer in a subject, comprising:
administering to a subject having cancer or at risk for cancer an effective amount of one or more binding agents that recognize one or more cell surface markers specific for M2-Tumor Associated Macrophage (TAM), wherein the effective amount of the binding agents is sufficient to treat or prevent the cancer.

2. A method selected from the group consisting of:
a method of reducing tumor associated macrophage density in a tumor of a subject comprising: administering to a subject having a tumor an effective amount of one or more binding agents that recognize one or more cell surface markers specific for M2-Tumor Associated Macrophages (TAMs), wherein the effective amount of the one or more binding agents is sufficient to reduce the density of tumor associated macrophages in the tumor of the subject;
a method of staging a tumor in a subject, comprising determining the presence of M2-Tumor Associated Macrophages (TAMs) in the subject; and
a method of diagnosing or predicting the progression of cancer in a subject, comprising determining the presence of M2 Tumor Associated Macrophages (TAMs) in the subject.

3-4. (canceled)

5. The method of claim 2, wherein the determining step comprises contacting a sample of cells from the subject with one or more binding agents that recognize one or more cell surface markers specific for M2-Tumor Associated Macrophage (TAM), and identifying cells recognized by the binding agents.

6. The method of claim 1, wherein the cell surface marker specific for M2-TAM is selected from the group consisting of: CD206 [mannose receptor], IL-4r, IL-1ra, decoy IL-1rII, IL-10r, CD23, macrophage scavenging receptors A and B, Ym-1, Ym-2, Low density receptor-related protein 1 (LRP1), IL-6r, CXCR1/2, CD136, CD14, CD1a, CD1b, CD93, CD226, (FcγR) and PD-L1.

7. The method of claim 1, wherein the binding agent is coupled to an imaging agent.

8. The method of claim 1, wherein the M2-TAM binding agent is an antibody, or an antigen binding fragment thereof.

9. The method of claim 8, wherein the antibody is a bispecific antibody, a trispecific antibody, an antibody with greater than three different specificities, or an antigen-binding fragment thereof.

10. The method of claim 8, wherein the antibody is conjugated to an additional agent, optionally a toxic agent, optionally a chemotherapeutic drug.

11-12. (canceled)

13. The method of claim 1, wherein the M2-TAM binding agent is a nanoparticle or a liposome, optionally wherein the nanoparticle is coated with a M2-TAM cell surface receptor ligand.

14. (canceled)

15. The method of claim 13, wherein the M2-TAM cell surface receptor ligand is selected from the group consisting of: CD206 [mannose receptor], IL-4r, IL-1ra, decoy IL-1rII, IL-10r, CD23, macrophage scavenging receptors A and B, Ym-1, Ym-2, Low density receptor-related protein 1 (LRP1), IL-6r, CXCR1/2, CD136, CD14, CD1a, CD1b, CD93, CD226, (FcγR) and PD-L1.

16. The method of claim 15, wherein the cell surface receptor ligand is coupled to an imaging agent.

17. The method of claim 13, wherein the nanoparticle or liposome comprises an additional agent, optionally a toxic agent, optionally a chemotherapeutic drug.

18-19. (canceled)

20. The method of claim 17, wherein the toxic agent is a bisphosphonate compound.

21. The method of claim 17, wherein the toxic agent is a radioactive compound.

22. A composition comprising a particle comprising one or more toxic agents and a M2-TAM specific targeting peptide bound to a surface on the particle.

23. The composition of claim 22, wherein the particle is a nanoparticle.

24. The composition of claim 22, wherein the M2-TAM specific targeting peptide is selected from the group consisting of: CD206 [mannose receptor], IL-4r, IL-1ra, decoy IL-1rII, IL-10r, CD23, macrophage scavenging receptors A and B, Ym-1, Ym-2, Low density receptor-related protein 1 (LRP1), IL-6r, CXCR1/2, CD136, CD14, CD1a, CD1b, CD93, CD226, (FcγR) and PD-L1.

25. The composition of claim 22, wherein the toxic agent is a chemotherapeutic drug.

26. The composition of claim 22, wherein the toxic agent is a bisphosphonate compound.

27. The composition of claim 22, wherein the toxic agent is a radioactive compound.
